(19) United States
(12) Patent Application Publication
(10) Pub. No.: US 2019/0231689 A1
(43) Pub. Date: Aug. 1, 2019

(54) Amphiphilic Block Copolymer Composition With Enhanced Micelle Stability

(71) Applicant: SAMYANG BIOPHARMACEUTICALS CORPORATION, Seoul (KR)

(72) Inventors: Bum Chan MIN, Seoul (KR); Bong Oh KIM, Daejeon (KR); Yil Woong YI, Daejeon (KR); Suk Yen KO, Gyeonggi-Do (KR); Gyeong Hae KIM, Gyeonggi-do (KR); Hye Rim KIM, Gyeonggi-do (KR); Yoo Jeong YOON, Gyeongsangnam-do (KR); June Hee LEE, Gyeonggi-do (KR); Hye Jin JANG, Gyeonggi-Do (KR); Ho Joon CHOI, Seoul (KR)

(73) Assignee: SAMYANG BIOPHARMACEUTICALS CORPORATION

(21) Appl. No.: 15/938,116

(22) Filed: Mar. 28, 2018

(30) Foreign Application Priority Data
Jan. 29, 2018 (KR) 10-2018-0010825

(51) Int. Cl. A61K 9/107; C08G 81/00; C08G 63/08; C08G 65/08; A61K 47/34

(52) U.S. Cl. CPC A61K 9/1075; C08G 81/00; A61K 47/34; C08G 65/08; C08G 63/08

(57) ABSTRACT

A composition comprising an A-B type diblock copolymer or a B-A-B or A-B-A type triblock copolymer with enhanced micelle stability, wherein A is a hydrophilic block and B is a hydrophobic block, and a polymeric micelle composition, a drug carrier composition and a pharmaceutical composition comprising the same are provided.

### CROSS-REFERENCE TO RELATED APPLICATION

This application claims the benefit of earlier filing date and right of priority to Korean Patent Application No. 10-2018-0010825, filed 29 Jan. 2018, the contents of which are incorporated by reference herein in its entirety.

### FIELD

The present disclosure relates to an amphiphilic block copolymer composition with enhanced micelle stability, and more specifically, a composition of an A-B type diblock copolymer or a B-A-B or A-B-A type triblock copolymer, wherein A is a hydrophilic block and B is a hydrophobic block.

### BACKGROUND

Amphiphilic block copolymer (also referred to as “polymer” hereinafter) is recognized as a very important component in the field of nanodrugs. According to the recently enacted regulation, among the components of a pharmaceutical composition, evaluation of excipients is emerging as a very important factor, in addition to the active pharmaceutical ingredient. Various physicochemical analysis methods have been used as the evaluation means. Because amphiphilic block copolymer form micelles which are eventually used in the final formulation, it is important to evaluate micellar properties such as stability during use.

In order to evaluate the stability of micelles, the polymer is dissolved in water at a pre-determined concentration and properties such as particle diameter can be measured over time at room temperature. It is known that if polymeric micelles prepared from amphiphilic block copolymers are left at room temperature, new micelles are formed with increased size relative to the initially formed micelles through the procedure called secondary aggregation. Secondary aggregation of micelles is a common phenomenon, and if polymeric micelles prepared by a conventional method are left as above, occurrence of the secondary aggregation can be observed easily. Since the polymers form micelles which are used in a pharmaceutical composition, micellar properties such as secondary aggregation can seriously affect the stability of the pharmaceutical composition itself.

Secondary aggregation may have seriously adverse effects on the drug quality because it can cause unexpected side effects during storage and clinical application. It can cause the precipitation of an active pharmaceutical ingredient, clogging of injection needle when used as an injection formulation, and the like. Further, increase of micelle size can cause unexpected toxicity, such as immunotoxicity.

Therefore, it is important to prepare a stable polymeric micelle product with no aggregation even if it is left after the micelle formation. Preparation of a polymer with enhanced micelle stability is very important in order to secure the stability of the polymeric micelle product.

### SUMMARY

In an embodiment, the present disclosure provides an amphiphilic block copolymer composition with enhanced micelle stability.

In another embodiment, there is provided a polymeric micelle composition comprising the amphiphilic block copolymer composition.

In yet another embodiment, there is provided a drug carrier composition comprising the amphiphilic block copolymer composition.

In yet another embodiment, there is provided a pharmaceutical composition comprising the amphiphilic block copolymer composition and one or more drugs.

One aspect of the present invention relates to an amphiphilic block copolymer composition comprising an A-B type diblock amphiphilic block copolymer or a B-A-B or A-B-A type triblock amphiphilic block copolymer, wherein A is a hydrophilic block and B is a hydrophobic block, wherein the hydrophilic block (A) is polyalkylene glycol and the hydrophobic block (B) is poly-D,L-lactide or a poly-D,L-lactide polyglycolide copolymer; wherein, in a ¹H NMR (500 MHz, CDCl₃) spectrum of the amphiphilic block copolymer, the area of peaks on the left side of a vertical line passing through a trough to the left of the fifth peak from the right among the multiple peaks between 5.10 ppm and 5.30 ppm comprises about 30% or greater of the total peak area of the multiple peaks between 5.10 ppm and 5.30 ppm.

Alternatively, the vertical line may pass through the valley between the two (2) peaks appearing between 5.18 ppm and 5.20 ppm to divide the total peaks between 5.10 ppm and 5.30 ppm into a left side and a right side.

Another aspect of the present invention relates to a polymeric micelle composition comprising the amphiphilic block copolymer composition.

A further aspect of the present invention relates to a drug carrier composition comprising the amphiphilic block copolymer composition.

A still further aspect of the present invention relates to a pharmaceutical composition comprising the amphiphilic block copolymer composition and one or more drugs.

The amphiphilic block copolymer composition according to one aspect of the present invention provides excellent stability to micelles compared with conventional polymer compositions, and so does not generate aggregation even if left for an extended period of time after the micelle formation. The amphiphilic block copolymer composition can be used to prepare a stable polymeric micelle product, and the like which does not generate aggregation even if left for an extended period of time during the use.

### BRIEF EXPLANATION OF THE DRAWINGS

FIG. 1 is ¹H NMR (500 MHz, CDCl₃) spectrum of monomethoxy polyethylene glycol-poly-D,L-lactide (mPEG-PLA) polymer.

FIG. 2 is an enlarged spectrum of the peak (assignment L) corresponding to the methine hydrogen nucleus of the —CH(CH3)— part in the mPEG-PLA polymer.

FIGS. 3A and 3B show the size distributions of the particles before and after, respectively, a period of time following the formation of polymeric micelles formed by dissolving mPEG-PLA in an aqueous media.

### DETAILED DESCRIPTION

In one embodiment, the present disclosure provides an amphiphilic block copolymer composition without occurrence of secondary aggregation after the micelle formation.

The polymer composition according to the embodiment above is distinguished from conventional polymer compositions, and can be defined by a characteristic ¹H NMR spectrum.

In an embodiment, the amphiphilic block copolymer composition is an amphiphilic block copolymer composition comprising an A-B type diblock amphiphilic block copolymer in or a B-A-B or A-B-A type triblock amphiphilic block copolymer, wherein A is a hydrophilic block and B is a hydrophobic block, wherein the hydrophilic block (A) is polyalkylene glycol and the hydrophobic block (B) is poly-D,L-lactide or a poly-D,L-lactide polyglycolide copolymer; wherein, in a ¹H NMR (500 MHz, CDCl₃) spectrum of the amphiphilic block copolymer, the area of peaks on the left side of a vertical line passing through the trough to the left of the fifth peak from right among the multiple peaks between 5.10 ppm and 5.30 ppm comprises about 30% or greater of the total area of peaks between 5.10 ppm and 5.30 ppm.

In the ¹H NMR (500 MHz, CDCl₃) spectrum, the range between 5.10 ppm and 5.30 ppm corresponds to the methine hydrogen nucleus of the —CH(CH3)— part of the polymer.

Alternatively, the vertical line may pass through the valley between the two (2) peaks appearing between 5.18 ppm and 5.20 ppm to divide the total peaks between 5.10 ppm and 5.30 ppm into a left side and a right side.

In a particular embodiment, based on the vertical line, the area of the peaks existing on the left side may comprise about 31% or greater, for example, such as about 31% to about 35%, of the total peak area (100%) between 5.10 ppm and 5.30 ppm. In another particular embodiment, the area of the peaks existing on the left side of the vertical line comprises about 32% or greater, for example, such as about 32% to about 35%, of the total peak area (100%) between 5.10 ppm and 5.30 ppm.

In another particular embodiment, the hydrophilic block (A) may be a polyethylene glycol or monomethoxypolyethylene glycol.

In another particular embodiment, the hydrophobic block (B) may be poly-D,L-lactide.

In another particular embodiment, the hydrophilic block (A) may be polyethylene glycol or monomethoxypolyethylene glycol and the hydrophobic block (B) may be poly-D,L-lactide.

In another particular embodiment, the hydrophilic block (A) may be monomethoxypolyethylene glycol and the hydrophobic block (B) may be poly-D,L-lactide.

In another particular embodiment, the amphiphilic block copolymer may be an A-B type diblock copolymer.

In another particular embodiment, the amphiphilic block copolymer may have a number average molecular weight of about 1,000 to about 50,000 Daltons. In some other embodiments, the number average molecular weight is about 1,000 to about 20,000 Daltons, or about 1,000 to about 6,000 Daltons. For example, the number average molecular weight of the amphiphilic block copolymer may be about 1,430 to about 6,000 Daltons. In some other embodiments, the number average molecular weight is about 1,500 to about 6,000 Daltons, or about 2,000 to about 6,000 Daltons.

In another particular embodiment, the amphiphilic block copolymer may comprise the hydrophilic block (A) in an amount of about 20 to about 95% by weight, about 40 to about 95% by weight, about 50 to about 70% by weight, or about 50 to about 60% by weight, based on the total weight of the block copolymer.

In another particular embodiment, the amphiphilic block copolymer may satisfy the above number average molecular weight range of the amphiphilic block copolymer and the above amount of the hydrophilic block (A) in the amphiphilic block copolymer at the same time.

In another particular embodiment, the hydrophilic block (A) may have a number average molecular weight of about 1,000 to about 30,000 Daltons. In some other embodiments, the number average molecular weight is about 1,000 to about 10,000 Daltons, about 1,000 to about 3,000 Daltons, or about 1,000 to about 2,500 Daltons.

In another particular embodiment, the hydrophobic block (B) may have a number average molecular weight of about 1,000 to about 30,000 Daltons. In some other embodiments, the number average molecular weight is about 1,000 to about 10,000 Daltons, about 1,000 to about 3,000 Daltons, or about 1,000 to about 2,500 Daltons.

The amphiphilic block copolymer composition according to one aspect of the present invention may be prepared as a polymeric micelle composition.

The amphiphilic block copolymer composition according to one aspect of the present invention may be used as a drug carrier composition.

The amphiphilic block copolymer composition according to one aspect of the present invention, together with one or more drugs, may be prepared as a pharmaceutical composition. For example, it may be prepared as a pharmaceutical composition wherein a poorly water-soluble drug is contained in a polymeric micelle formed by the amphiphilic block copolymer composition. Specifically, it may be prepared as a pharmaceutical composition wherein a poorly water-soluble drug is physically encapsulated within the hydrophobic core of the polymeric micelle.

The pharmaceutical composition may be prepared according to a method conventionally known in this field of art. For example, the pharmaceutical composition may be prepared by agitation, heating, ultrasonic treatment, solvent evaporation, dialysis or the like without limitation.

The poorly water-soluble drug may be a drug having solubility in water of 50 mg/mL or less. The poorly water-soluble drug includes, for example, an antineoplastic agent, an antibiotic, an anti-inflammatory agent, an analgesic, a hormone, an agent for treating hypertension, an agent for treating diabetes, an agent for treating hyperlipidemia, an antiviral agent, an agent for treating Parkinson's disease, an agent for treating dementia, an antiemetic agent, an immunosuppressant, an agent for treating ulcer, an agent for treating constipation, an antimalarial agent, and the like.

In an embodiment, the poorly water-soluble drug may be a drug having a high reduction rate in blood when administered into human body. Examples of the drug include an antineoplastic agent such as paclitaxel, camptothecin, etoposide, doxorubicin, daunorubicin, idarubicin, Ara-C, etc., an immunosuppressant such as cyclosporine, etc., a steroid hormone drug such as testosterone, estradiol, estrogen, progesterone, triamcinolone acetate, dexamethasone, etc., an anti-inflammatory agent such as tenoxicam, piroxicam, indomethacin, ibuprofen, COX-II inhibitor, etc.

In order to solubilize the poorly water-soluble drug in the polymeric micelle composition, the poorly water-soluble drug may be encapsulated, for example, in an amount of about 0.1 to about 30 parts by weight, based on 100 parts by weight of the polymer and the drug.

The pharmaceutical composition may be administered orally or parenterally. In case of parenteral administration, the poorly water-soluble drug may be administered via intravenous, intramuscular, subcutaneous, intraperitoneal, transnasal, intrarectal, intraocular, or intrapulmonary route, or the like.

The present invention is explained in more detail by the following examples. These examples illustrate various embodiments of the present invention. The scope of the present invention is not limited by the examples in any manner.

### EXAMPLE

### Example

### Synthesis of a Diblock Copolymer Consisting of monomethoxypolyethylene glycol and oly-D,L-lactide (mPEG-PLA)

100 g of monomethoxypolyethylene glycol (mPEG, number average molecular weight=2,000) was fed into a 250-ml round-bottom flask equipped with an agitator, and agitated at 120° C. under vacuum condition (0.2 torr) for 10 hours to remove moisture. 0.1 g of stannous octoate (Sn(Oct)₂) dissolved in toluene (200 μl) was added in the reaction flask, and the mixture was further agitated under vacuum condition for 1 hour to distill and remove the toluene.

Then, 100 g of D,L-lactide, which had been dried at room temperature under vacuum condition (0.1 torr) for 10 hours, was added and agitated under nitrogen atmosphere for dissolution. After D,L-lactide was dissolved completely, the reactor was tightly sealed and the polymerization reaction was conducted at 120° C. for 10 hours. After the reaction was completed, 192 g of the unpurified diblock copolymer (mPEG-PLA polymer) was obtained (number average molecular weight: 3,685 Daltons).

### Comparative Example

### Synthesis of a Diblock Copolymer Consisting of monomethoxypolyethylene glycol and poly-D,L-lactide (mPEG-PLA)

100 g of monomethoxypolyethylene glycol (mPEG, number average molecular weight=2,000) was fed into a 250-ml round-bottom flask equipped with an agitator, and agitated at 120° C. under vacuum condition (0.5 torr) for 2 hours to remove moisture. 0.1 g of stannous octoate (Sn(Oct)₂) dissolved in toluene (200 μl) was added in the reaction flask, and the mixture was further agitated under vacuum condition for 1 hour to distill and remove the toluene.

Then, 100 g of D,L-lactide, which had been dried at room temperature under vacuum condition (0.5 torr) for 2 hours, was added and agitated under nitrogen atmosphere for dissolution. After D,L-lactide was dissolved completely, the reactor was tightly sealed and the polymerization reaction was conducted at 120° C. for 10 hours.

After the reaction was completed, 188 g of the unpurified diblock copolymer (mPEG-PLA polymer) was obtained (number average molecular weight: 3,740 Daltons).

### Experimental Example 1

### ¹H-NMR Measurement and L Ratio Calculation of mPEG-PLA

Each of the polymers obtained in the above Example and Comparative Example was analyzed with ¹H NMR spectroscopy. The instrument and sample pretreatment conditions used for the NMR measurement are as follows:

Instrument: Bruker Ascend 500 (500 MHz NMR)

NMR tube: 5mm tube

NMR solvent: CDC13

Sample concentration: 1 mg/mL

NMR sample volume: 0.7 mL

Measurement temperature: 27° C.

For the NMR tube, tube cap and pipet for preparing the NMR sample, new products were used, and prior to use, they were dried in a desiccator for 24 hours or more so that no moisture remained. For the NMR solvent (CDCl₃), 0.75 mL ampule with a purity of 99.95% or higher (Aldrich) was used immediately after opening for preparing the NMR sample.

The NMR measurement was conducted according to the ASTM method (ASTM E2977-15). Additionally the following conditions for measurement and data processing were used. All measurements and data processing were performed using software originally installed on the Bruker Ascend 500 instrument.

Time Domain Data Size: 60K

Acquisition Time: 3 sec

Relaxation Delay: 3 sec

Number of Scan: 128

Frequency Domain Data Size: 60K

Window Function: Exponential multiplication

Line Broadening Factor: 0.3

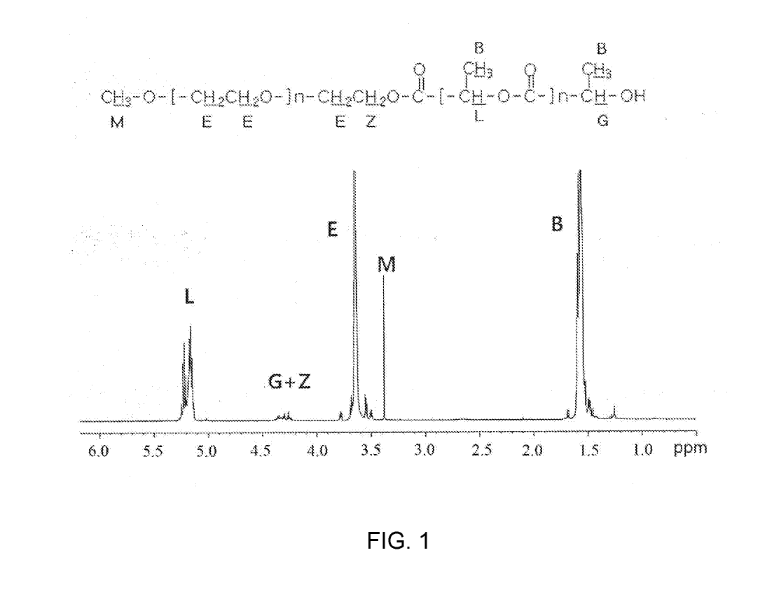

FIG. 1 is ¹H NMR (500 MHz, CDCl₃) spectrum of mPEG-PLA polymer. In FIG. 1, the peaks corresponding to the hydrogens in the components of the polymer are shown in association with the molecular structure of the polymer.

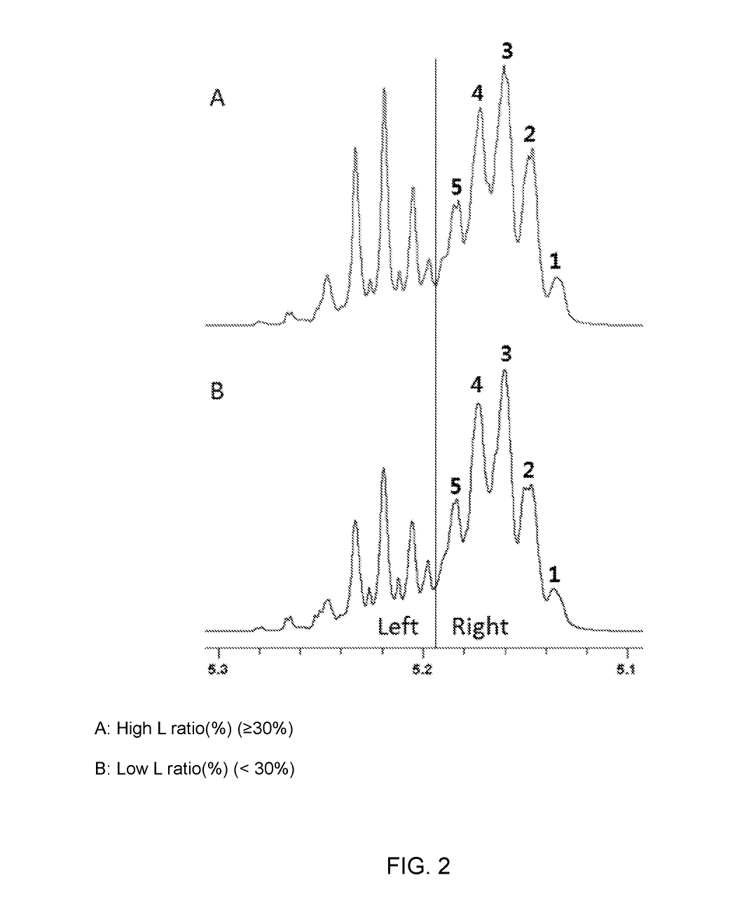

FIG. 2 is an enlarged spectrum of the peak corresponding to the methine hydrogen nucleus of the −CH(CH3)— part in mPEG-PLA polymer. This is the peak assigned as L corresponding to around 5.2 ppm on the spectrum. As shown in FIG. 2, the NMR peaks in this part are shown as complex multiple lines. The peak shapes of the polymer of Example in this part are different from those of the polymer of Comparative Example. That is, the polymer of Example showed the shapes presented in A (top) whereas the polymer of Comparative Example showed the shapes presented in B (bottom). The most important feature is that there is a difference in the relative intensity of the left part compared to the right part of the multiple peaks between 5.10 ppm and 5.30 ppm between the polymer of the Example and the polymer of the Comparative Example.

In the ¹H NMR (500 MHz, CDCl₃) spectra of the polymers measured under the conditions of Experimental Example 1, the area of the peaks to the left of the vertical line passing through the trough to the left of the fifth peak from the right of the multiple peaks between 5.10 ppm and 5.30 ppm, which corresponds to the methine hydrogen nucleus of the —CH(CH3)— part of the polymer is different between the polymer of Example and the polymer of Comparative Example. This is defined as L ratio.

L ratio=(Peak area of the left side/Total peak area)×100

Peak area of the left side: The area of the peaks existing on the left side of the vertical line passing through the trough to the left of the fifth peak from the right of the multiple peaks between 5.10 ppm and 5.30 ppm (in another expression, the area of the peaks existing on the left side of the vertical line passing through the valley between the two (2) peaks appearing between 5.18 ppm and 5.20 ppm)

Total peak area: The sum of all peak areas appearing in 5.10 ppm to 5.30 ppm

The L ratio in the polymer of the Example was higher than the L ratio in the polymer of the Comparative Example (Table 1).

### Experimental Example 2

### Micelle Stability Test for mPEG-PLA

For several polymers prepared by the same method as Example and Comparative Example, the stability tests were conducted after micelle formation in an aqueous solution.

Each of the samples prepared as in the Example and the Comparative Example was dissolved in distilled water at 1 mg/mL concentration, then left at 30° C. and 50° C. for 3 days. The change in particle size according to the lapse of time was measured (referred to as ‘micelle stability test’ hereinafter). For this, a DLS (Dynamic Light Scattering) instrument capable of measuring the particle size distribution and the average particle diameter was used.

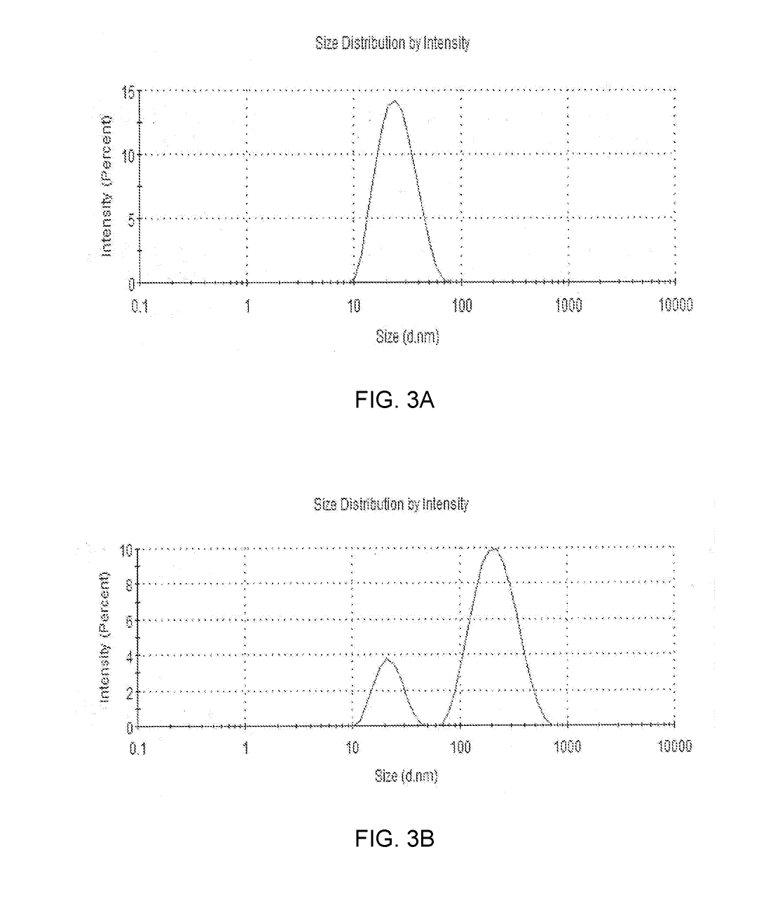

The particle size distributions before/after the micelle stability test are shown in FIG. 3. The distribution shown in FIG. 3A is a typical result when preparing a polymer, immediately after dissolving it in water, and measuring the average particle diameter. The particles showed a particle size distribution having a single peak below 20 nm.

However, at the end of the micelle stability test, the particle size distribution and the average particle diameter of the polymer of Example showed different pattern from those of the polymer of Comparative Example. In case of the polymer of the Example, even at the end of the micelle stability test, the particle size distribution maintained a single peak of the same particle size shown in FIG. 3A. However, in case of the polymer of the Comparative Example, the particle size distribution is in FIG. 3B. That is, in the particle size distribution measurement at the end of the micelle stability test with the polymer of Comparative Example, particles below 20 nm still existed but particles having a larger particle diameter of about 200 nm were generated.

In general, micelle stability can be evaluated on the basis of the average diameter of the particles shown in the particle size distribution. Since the average diameter of micelles becomes larger with the lapse of time, if the particle size of micelles is larger than a specific average particle diameter at a specific time, the stability can be recognized as insufficient. In the present Experimental Example (micelle stability test), the stability evaluation was based on the average particle diameter after 2 days in case of the test at 30° C., and the average particle diameter after 1 day in case of the test at 50° C. In both tests at 30° C. and 50° C., the polymer having an average particle diameter greater than 30 nm is defined as “Fail”, and the polymer maintaining the average particle diameter of 30 nm or less even after the test is defined as “Pass.” The average particle diameter and the Pass/Fail result of each polymer with the lapse of time in the micelle stability test are shown in Table 1, along with the L ratio.

TABLE 1
Results of the micelle stability tests at 30° C. and 50° C.

| | | | |
|---|---|---|---|
| Polymer | Average particle | | L |
| batch | diameter (nm) | Pass/Fail | ratio (%) |

Results of the micelle stability test at 30° C.

| | | | | |
|---|---|---|---|---|
| Example | A | 18 | Pass | 33 |
| | B | 18 | Pass | 32 |
| Comparative | C | 35 | Fail | 29 |
| Example | D | 36 | Fail | 28 |

Results of the micelle stability test at 50° C.

| | | | | |
|---|---|---|---|---|
| Example | A | 20 | Pass | 33 |
| | B | 17 | Pass | 32 |
| Comparative | C | 46 | Fail | 29 |
| Example | D | 49 | Fail | 28 |

As shown in Table 1, it can be confirmed that in order to be considered as “Pass”, the L ratio should be at least 30%, particularly 32%, or higher. That is, it can be confirmed that only the polymer compositions—wherein in a ¹H NMR (500 MHz, CDCl₃) spectrum measured under the conditions of Experimental Example 1, the area of the peaks to the left of the vertical line passing through the trough to the left of the fifth peak from the right, when numbering the multiple peaks between 5.10 ppm and 5.30 ppm corresponding to the methine hydrogen nucleus of the —CH(CH3)— part of the polymer comprises 30% or greater of the total peak area (the sum of all peak areas between 5.10 ppm and5.30 ppm) (alternatively, the vertical line may pass through the valley between the two (2) peaks appearing between 5.18 ppm and 5.20 ppm to divide the total peaks between 5.10 ppm and 5.30 ppm into a left side and a right side)—can satisfy the requirements to “Pass” the micelle stability test.

What is claimed is:

1. An amphiphilic block copolymer composition comprising an A-B type diblock amphiphilic block copolymer or a B-A-B or A-B-A type triblock amphiphilic block copolymer,
wherein A is a hydrophilic block and B is a hydrophobic block, wherein the hydrophilic block (A) is polyalkylene glycol and the hydrophobic block (B) is poly-D,L-lactide or a poly-D,L-lactide polyglycolide copolymer; and
wherein, in a ¹H NMR (500 MHz, CDCl₃) spectrum of the amphiphilic block copolymer, the area of the peaks to the left of a vertical line passing through a trough to the left of the fifth peak from the right among the multiple peaks between 5.10 ppm and 5.30 ppm, comprises about 30% or greater of the total peak area.

2. The amphiphilic block copolymer composition according to claim 1, wherein the area of the peaks existing on the left side of the vertical line comprises about 32% or greater of the total peak area.

3. The amphiphilic block copolymer composition according to claim 1, wherein the hydrophilic block (A) is polyethylene glycol or monomethoxypolyethylene glycol.

4. The amphiphilic block copolymer composition according to claim 1, wherein the hydrophobic block (B) is poly-D,L-lactide.

5. The amphiphilic block copolymer composition according to claim 1, wherein the hydrophilic block (A) is polyethylene glycol or monomethoxypolyethylene glycol and the hydrophobic block (B) is poly-D,L-lactide.

6. The amphiphilic block copolymer composition according to claim 5, wherein the hydrophilic block (A) is monomethoxypolyethylene glycol and the hydrophobic block (B) is poly-D,L-lactide.

7. The amphiphilic block copolymer composition according to claim 1, wherein the amphiphilic block copolymer is an A-B type diblock copolymer.

8. The amphiphilic block copolymer composition according to claim 1, wherein the amphiphilic block copolymer comprises the hydrophilic block (A) in an amount of about 20 to about 95% by weight, based on the total weight of the block copolymer.

9. The amphiphilic block copolymer composition according to claim 8, wherein the amphiphilic block copolymer comprises the hydrophilic block (A) in an amount of about 40 to about 95% by weight, based on the total weight of the block copolymer.

10. The amphiphilic block copolymer composition according to claim 9, wherein the amphiphilic block copolymer comprises the hydrophilic block (A) in an amount of about 50 to about 70% by weight, based on the total weight of the block copolymer.

11. The amphiphilic block copolymer composition according to claim 1, wherein the amphiphilic block copolymer has a number average molecular weight of about 1,000 to about 50,000 Daltons.

12. The amphiphilic block copolymer composition according to claim 11, wherein the amphiphilic block copolymer has a number average molecular weight of about 1,000 to about 20,000 Daltons.

13. The amphiphilic block copolymer composition according to claim 12, wherein the amphiphilic block copolymer has a number average molecular weight of about 1,500 to about 6,000 Daltons.

14. A polymeric micelle composition, comprising an amphiphilic block copolymer composition of claim 1.

15. A drug carrier composition, comprising an amphiphilic block copolymer composition of claim 1.

16. A pharmaceutical composition, comprising an amphiphilic block copolymer composition of claim 1 and one or more drugs.

17. A polymeric micelle composition, comprising an amphiphilic block copolymer composition of claim 2.

18. A polymeric micelle composition, comprising an amphiphilic block copolymer composition of claim 6.

19. A polymeric micelle composition, comprising an amphiphilic block copolymer composition of claim 10.

20. A polymeric micelle composition comprising an amphiphilic block copolymer composition of claim 13.
